(19) United States
(12) Patent Application Publication
(10) Pub. No.: US 2006/0025848 A1
(43) Pub. Date: Feb. 2, 2006

(54) Medical device having a coating layer with structural elements therein and method of making the same

(76) Inventors: Jan Weber, Maple Grove, MN (US); Tom Holman, Princeton, MN (US)

Correspondence Address: JONES DAY, 222 EAST 41ST ST, NEW YORK, NY, 10017, US

(21) Appl. No.: 10/902,747

(22) Filed: Jul. 29, 2004

(51) Int. Cl. A61F 2/06

(52) U.S. Cl. 623/1.15; 623/1.42; 424/426

(57) ABSTRACT

The invention pertains to coated medical devices, such as stents and balloon catheters, for delivering a biologically active material to body tissue of a patient. The medical device has a coating layer comprising a biocompatible polymer, non-polymer material, or biologically active material disposed on its surface, and at least one structural element embedded within the coating layer. The structural elements reduce the compressibility of the coating layer. The structural element may be any shape or configuration. A biologically active material may be dispersed within the coating layer or structural elements. Methods for making such medical devices are also disclosed.

### FIELD OF THE INVENTION

This invention relates generally to a medical device having a coating layer disposed on at least a portion of the surface of the medical device. More particularly, this invention is directed to a medical device having a coating layer disposed on at least a portion of its surface and at least one structural element embedded in the coating. The structural element reduces the compressibility of the coating layer. Also, the coating layer is capable of delivering a biologically active material to a desired location within the body of a patient. The invention is also directed to a method for manufacturing such a coated medical device.

### BACKGROUND OF THE INVENTION

A variety of medical conditions are commonly treated by introducing an insertable or implantable medical device in to the body. In many instances, the medical device is coated with a material, such as a polymer, which is able to release a biologically active agent. For example, various types of drug-coated stents have been used for localized de livery of drugs to a body lumen. See, e.g., U.S. Pat. No. 6,099,562 to Ding et al.

Existing coatings on such medical devices may be compressible or deformed by sheer forces. Thus, if compressive forces are applied to the coatings, the thickness of the coatings will decrease. Such decrease in coating thickness may lead to certain potential disadvantages. For example, during delivery of the medical device, or even after delivery of the device, shear forces exerted on the device may cause a premature release of the biologically active material from the compressed coating. Thus, it may be difficult to control when or at what rate the biologically active agent is released from the coating. In other instances, compressive forces may remove some of the coating from the medical device surface. Such displacement or removal of the coating is undesirable. For instance, the targeted site may not receive the adequate amount of biologically active agent or healthy tissue be unnecessarily exposed to the biologically active agent. Furthermore, sheer forces applied on the coating may push the coating toward one side and form an uneven coating which may be thicker at one region and thinner at another region. At the thicker region, there may be overloading of drugs whereas at the thinner region there may be underloading of drugs.

One possible way to prevent or reduce the undesired compression or sheering force of the coating layer is to coat the medical device with a less compressible or sheerable coating, such as a rigid coating. A more rigid coating however, may have other potential disadvantages. For example, a more rigid coating may not be able to accommodate a high concentration of biologically active material. Thus, to form a coating with a better affinity for a biologically active material, a less rigid polymeric material may be desirable. Other possible disadvantages of a more rigid coating include a change in release rate as compared to a less rigid coating; or that the rigid coating may have a greater susceptibility to stress induced cracks or cracks caused by dilation forces of the medical device, resulting in a sudden exposure of biological agent.

Therefore, there is a need for a medical device having a coating that can incorporate an adequate amount of biologically active agent and also have reduced compressibility, i.e. the extent to which the coating layer height or thickness is reduced by a compression force applied to the coating layer during delivery and implantation. Another need is for a medical device, such as a balloon catheter, having a protective coating to prevent tearing of the balloon when the balloon catheter encounters obstacles, such as calcification, in the body of a patient. There is also a need for a method for making such medical device.

### SUMMARY OF THE INVENTION

These and other objectives are accomplished by the present invention. To achieve the aforementioned objectives, we have invented a coated medical device, such as a stent, or a balloon catheter, comprising: a medical device having a surface and a coating layer disposed on at least a portion of the surface. The coating layer comprises a biocompatible polymer having at least one structural element embedded into the coating layer. The structural element reduces the compressibility of the coating layer. In another embodiment, the coating layer comprises a biocompatible non-polymeric material having at least one structural element embedded into the coating layer. In another embodiment, the coating layer comprises a biologically active material having at least one structural element embedded into the coating layer. The thickness of a coating layer, when a compression force is applied to the coating layer, can be any percentage of the thickness of the coating layer absent the compression force. For example, the thickness when the compression is applied can be at least about 10%, 20%, 30%, 40%, 50%, 60%, 70%, 80%, 85%, 90% or 95% of the thickness of the coating layer absent the compression force.

In certain embodiments, the structural elements may have a thickness or height that is greater, equal to or less than the thickness of the coating layer. In specific embodiments, the structural elements may have a height or thickness of at least 120%, 110%, 100%, 99%, 95%, 90%, 85%, 80%, 75%, 70%, 65%, 60%, 55%, 50%, 45%, 40%, 35%, 30%, 25%, 20%, 15%, 10% or 5% of the thickness of the coating layer containing the structural elements. It is also preferred that the polymeric material, the non-polymeric material, or the biologically active material coating has a first hardness and the structural element has a second hardness that is greater than the first hardness.

Preferably, the structural element comprises a metal, a ceramic, a polymer, or a biologically active material. The structural element may comprise a porous material or a biodegradable material, such as a polyelectrolyte biodegradable shell enclosing, for example, a crystallized drug.

A plurality of structural elements which are of the same or different shape may be embedded in the coating layer. In certain embodiments, at least two of the structural elements are interconnected. The structural element may be in contact with the device surface. Moreover, the structural element may be configured in a shape comprising, for example, a sphere, a shell, a disc, a rod, a strut, a rectangle, an oblique spheroid, a cube, a triangle, a pyramid, tripod, tetrahexdron, a matrix, a wire network, crosslinked microvolumes, which may be hard crystalline polymeric regions, within a polymer coating or combinations thereof. The structural element may also comprise more than one layer or combinations thereof. Further, when two or more coating layers are present, the structural element may reside in one or more of the coating layers. In certain embodiments, when compressed, the structural elements may create openings by pinching through an adjacent coating layer in which the structural elements are absent, thus allowing the drugs to be released through the openings.

In another embodiment, the coating layer comprises at least one biologically active material, such as an anti-thrombogenic agent, an anti-angiogenesis agent, an anti-proliferative agent, a growth factor, a radiochemical or combinations thereof. The anti-proliferative agent may include paclitaxel, a paclitaxel analogue, a paclitaxel derivative, or combinations thereof.

Also described herein is a coated stent for delivering a biologically active material to body tissue. The stent has a surface and a coating layer disposed on at least a portion of the surface. The coating layer comprises a biocompatible polymer, or non-polymeric material, a biologically active material and a plurality of structural elements embedded in the coating layer. The structural element reduces the compressibility of the coating layer.

Also described herein is another coated stent for delivering a biologically active material to body tissue. The stent has a surface and a coating layer disposed on at least a portion of the surface. The coating layer comprises a porous biocompatible polymer. In another embodiment, the coating layer comprises a biocompatible non-polymeric material. In another embodiment, the coating layer comprises a biologically active material. Embedded in the coating layer are a plurality of structural elements comprising a biologically active material. The structural elements reduce the compressibility of the coating layer and comprise a porous material having a second hardness, wherein the first hardness of the biocompatible polymer, non-polymeric material, or biologically active material, is less than the second hardness of the structural elements. The structural elements may comprise a second biologically active material. Also, the structural elements may comprise a ceramic.

In one embodiment, the coating layer comprises a non-polymeric material and embedded in the coating layer, a plurality of structural elements. In certain embodiments, the structural elements comprise a biologically active material.

In one embodiment, the coating layer comprises a biologically active material and embedded in the coating layer, a plurality of structural elements. In certain specific embodiments, the structural elements comprise a second biologically active material.

Also described herein is another coated stent for delivering a biologically active material to body tissue. The stent has a surface and a coating layer disposed on at least a portion of the surface. The coating layer comprises a porous biocompatible polymer and embedded in the coating layer, a plurality of structural elements comprising a biologically active material. The structural elements and the porous polymer have different hardness. When compressed, the structural elements release the biologically active material to the pores of the coating layer. In certain embodiments, the biocompatible polymer has hardness that is greater than that of the structural elements.

The present invention provides for a coated medical device in which the coating layer is more resistant to compressive forces or sheering forces. The coating provides protection to the underlying medical device. The invention provides a medical device in which the release time and rate of a biologically active material from the coating layer can be better controlled. Controlling the release rate is useful, for example, prior to the delivery and expansion of the medical device, a minimal release of biologically active material is required. Upon implantation and expansion of the medical device, there is a need for a larger amount of biologically active material. Also, the present invention provides for an efficient and effective method of manufacturing such a medical device.

### BRIEF DESCRIPTION OF THE DRAWINGS

FIG. 1 is a cross-sectional view of a medical device having at least one structural element embedded in a coating layer disposed on at least a portion of the device surface in which the coating layer contains a biologically active agent.

FIG. 2 is a cross-sectional view of a medical device having a coating layer disposed on at least a portion of the device surface and at least one structural element disposed on the surface, in which the structural element is not covered by the coating layer.

FIG. 3 is a cross-sectional view of a medical device having a coating layer disposed on at least a portion of the surface and at least one structural element embedded in the coating layer.

FIG. 4 is a cross-sectional view of a medical device having a coating comprising a plurality of coating layers and at least one structural element embedded in one of the coating layers.

FIG. 5A is a cross sectional view of a medical device devoid of structural elements and a coating layer of height h.

FIG. 5B is a cross sectional view of a medical device devoid of structural elements and a coating layer wherein the coating layer is compressed to a height of x.

FIG. 5C is a cross sectional view of a medical device having structural elements and a coating layer of height h.

FIG. 5D is a cross sectional view of a medical device having structural elements and a coating layer wherein the coating layer has been compressed to a height of y, wherein y is the height of the structural elements.

FIG. 6 is a cross-sectional view of a medical device having a coating layer disposed on at least a portion of the surface and at least one porous structural element embedded into the coating layer.

FIG. 7 is a cross-sectional view of a medical device having coating layer disposed on at least a portion of the surface and at least two interconnected structural elements embedded in the coating layer.

FIG. 8 is a cross-sectional view of a medical device having at least two interconnected structural elements that are disposed on the surface.

FIG. 9 is a cross-sectional view of a medical device having a coating layer disposed on at least a portion of the surface and structural elements of various shapes embedded into the polymer.

FIG. 10 is a cross-sectional view of a medical device having a coating layer disposed on at least a portion of the surface and a plurality of layered structural elements embedded in the coating layer.

FIG. 11 is a cross-sectional view of a medical device having a coating layer disposed on at least a portion of the surface and at least one structural element embedded in the polymer which is 50% of the thickness of the coating layer absent compression.

FIG. 12 is a cross-sectional view of a medical device having a coating layer disposed on at least a portion of the surface and structural elements of varying sizes embedded in the polymer.

### DETAILED DESCRIPTION

The coated medical devices of the present invention can be inserted and implanted in the body of a patient. Medical devices suitable for the present invention include, but are not limited to, stents, surgical staples, catheters, such as balloon catheters, central venous catheters, and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, septal defect devices, hemodialysis units, hemoperfusion units and plasmapheresis units.

Medical devices suitable for the present invention include those that have a tubular or cylindrical-like portion. The tubular portion of the medical device need not be completely cylindrical. For instance, the cross-section of the tubular portion can be any shape, such as rectangle, a triangle, etc., not just a circle. Such devices include, without limitation, stents, balloon catheters, and grafts. A bifurcated stent is also included among the medical devices which can be fabricated by the method of the present invention.

Medical devices that are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. In preferred embodiments, the stent suitable for the present invention is an Express stent. More preferably, the Express stent is an Express™ stent or an Express2™ stent.

Medical devices that are suitable for the present invention may be fabricated from metallic, ceramic, or polymeric materials, or a combination thereof. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Suitable ceramic materials include, but are not limited to, oxides, carbides, or nitrides of the transition elements such as titaniumoxides, hafnium oxides, iridiumoxides, chromium oxides, aluminum oxides, and zirconiumoxides. Silicon based materials, such as silica, may also be used.

The polymer(s) useful for forming the medical device should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

Medical devices may be coated or made with non-polymeric materials. Examples of useful non-polymeric materials include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides.

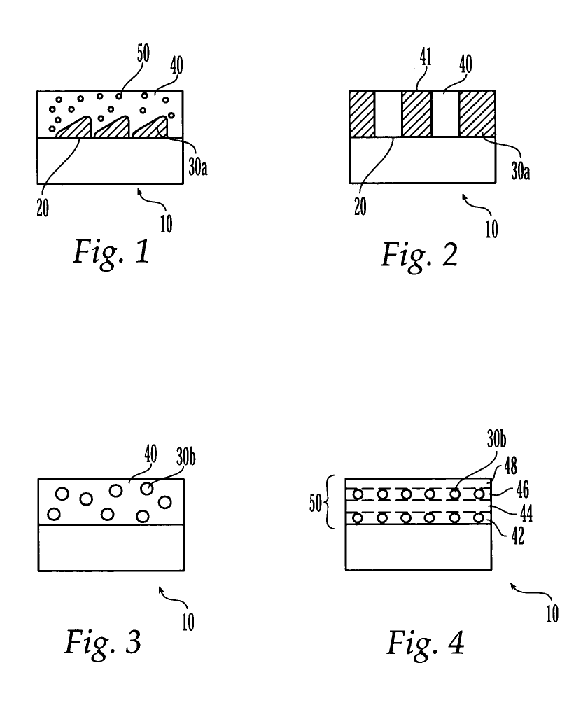

The medical device of the present invention has a surface and a coating layer disposed on the surface. The coating layer comprises at least one structural element, preferably a plurality of structural elements. The structural elements are embedded in the coating layer, i.e. the structural elements are at least partially surrounded by or in contact with the coating layer material. In some embodiments, at least some of the structural elements are completely surrounded by the coating material layer. In other embodiments, the coating layer material only partially surrounds the structural elements. FIG. 1 is a cross-sectional view of a portion of a medical device 10 having a surface 20, at least one structural element 30a disposed on the surface 20 and a coating layer 40 of coating layer material that partially surrounds the structural elements 30a. The coating layer 40 of this embodiment also includes a biologically active material 50.

FIG. 2 is a cross-sectional view of another embodiment of the present invention. In this embodiment a medical device 10 having a surface 20 is coated by a coating layer 40. At least one structural element 30a is disposed on the surface 20. The coating material of the coating layer 40 only partially surrounds the structural elements 30a so that the tops of the structural elements 41 are not covered by the coating layer material and the bottom portion of the structural element 30a is in contact with the surface 20.

Alternatively, as shown in FIG. 3, at least some of the structural elements 30b are completely surrounded by the coating layer 40 material.

FIG. 4 shows another embodiment in which the structural elements 30b are surrounded by the coating layer material. However, FIG. 4 depicts a coating 60 comprising four coating layers 42, 44, 46 and 48 in which two of the coating layers 42 and 46 include structural elements 30b. In alternate embodiments, all of the coating layers 42, 44, 46 and 48 of the coating 60 can include structural elements 30b.

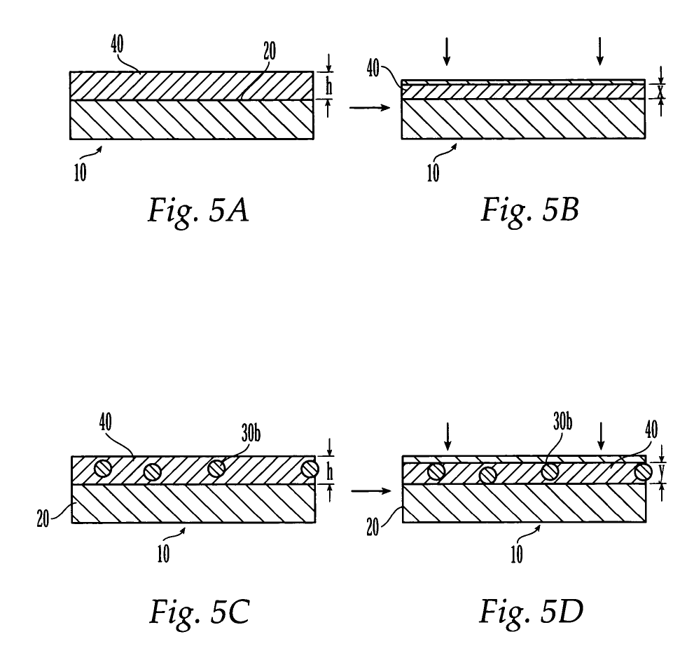

The structural elements reduce the compressibility of the coating layer, i.e., the ability of the thickness of the coating layer to be compressed or reduced in higher thickness by the compressive force. Generally, the structural elements are formed from a material that is less compressible or harder than the material(s) used to form the coating layer. In other words, the structural elements have a first hardness and the material used to form the coating layer, e.g. a polymer, has a second hardness that is less than the first hardness. As shown in FIGS. 5A-5D the inclusion of structural elements 30b in the coating layer 40 reduces the compressibility or maximum extent to which the coating layer height or thickness can be reduced by a given compression force. FIG. 5A shows a medical device 10 having a surface 20 and a coating layer 40 disposed on the surface 20. The coating layer 40 has a height or thickness h but does not include any structural elements. FIG. 5B shows a compressive force, which is indicated by the downward arrows, applied to the coating layer 40. The compressive force compresses the coating layer 40 and reduces its height from h to x.

FIG. 5C shows a coated medical device 10 similar to that shown in FIG. 5A. However, the coating layer 40 which has a height of h in FIG. 5C includes structural elements 30b. FIG. 5D shows the compressive force, which is indicated by the downward arrows, and which was applied to the coating layer 40 in FIG. 5B being applied to the coating layer 40. The compressive force compresses the coating layer 40 and reduces its height from h to y. The height y of the compressed coating layer 40 containing structural elements 30b shown in FIG. 5D is greater than the height x of the compressed coating layer 40 without structural elements shown in FIG. 5B. Therefore, these figures show that inclusion of structural elements reduces the compressibility of the coating layer 40.

In this application “compressive forces” or “compression force” refers to forces applied to the medical device 10 in all directions. This includes but is not limited to forces experienced by the medical device 10 upon introduction and deployment into the body lumen which may cause the coating layer 40 to be displaced, stripped or compacted. Compressive forces also include forces exerted on the medical device 10 when the medical device 10 reaches its destination which may cause the coating layer 40 to be compacted or deformed. Additionally, compressive forces can include manufacturing induced compression, as that resulting from crimping of the device or stent on a balloon.

Moreover, the compressive force applied to the coating can be one that is purposely applied to the coating. Since the amount of compression applied to the coating can affect the rate of biologically active material released from the coating, one can apply a certain predefined or predetermined amount of a compressive force to the coating to achieve the desired release rate. The compressive force may be applied through a balloon catheter. The thickness of a coating layer, when a compressive force is applied to the coating layer, can be any percentage of the thickness of the coating layer absent the compression force. For example, the thickness when the compression is applied can be at least about 10%, 20%, 30%, 40%, 50%, 60%, 70%, 80%, 85%, 90% or 95% of the thickness of the coating layer absent the compression force.

Furthermore, in one embodiment where the coating comprises more than one coating layer, structural elements can be placed in some but not all the coating layers (see FIG. 4). For example, structural elements can be placed in a first coating layer. A second coating layer is deposited over this first coating layer. This second coating layer does not include structural elements. A compression force can be applied to the coating so that the structural elements of the first coating layer pinch through the second coating layer to produce holes in the second coating layer to facilitate release of biologically active material from the coating.

In another embodiment, structural elements may be incorporated into a stent after the stent has been placed in the body of a patient. In a specific embodiment, the stent of the present invention may be a magnetic stent with a soft coating comprising a biologically active material on the surface of the stent. In another embodiment, the stent comprises a first and a second coating layer. The first coating layer is a soft coating layer which comprises a biologically active material. The second coating layer is another coating layer having a different softness than the first coating layer. An amount of magnetic nanoparticles may be injected in the blood stream of the patient in which a magnetic stent was implanted. The injected magnetic nanoparticles may be attracted by the implanted magnetic stent and migrate through the second coating layer of the magnetic stent. In certain embodiments, the magnetic nanoparticles may be deposited in the second coating layer or the first coating layer of the magnetic stent. Magnetic nanoparticles useful in this inventions are for example Cobalt alloys such as: Cobalt-palladium (Co—Pd) and Cobalt-platinum (Co—Pt), or Iron alloys such as Iron-Gold (Fe—Au), Iron-Chromium (Fe—Cr), Iron nitride (Fe—N), Iron oxide (Fe304), Iron-palladium (Fe—Pd), Iron-platinum (Fe—Pt).

Additionally, in one embodiment, the coating layer can comprise a porous coating material having a hardness that is greater than that of the structural elements embedded or placed in the coating layer. The structural elements include a biologically active material. When the coating layer is compressed, the structural elements are squeezed and the biologically active material of the structural elements are released into the pores of the porous coating material. The biologically active material is then released from the coating layer.

The structural elements can be made of many different materials. Suitable materials include, but are not limited to, the materials from which the medical device 10 is constructed as listed above. Also, the material of the structural elements may be porous or nonporous. Porous structural elements can be microporous, nanoporous or mesoporous.

Structural elements suitable for the present invention may be fabricated from metallic, ceramic, or polymeric materials, or a combination thereof. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. The structural element may also include parts made from other metals such as, for example, gold, platinum, or tungsten.

The polymeric material may be biostable. Also, the polymeric material may be biodegradable. Suitable polymeric materials include, but are not limited to, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, and Teflon. Suitable ceramic materials include, but are not limited to, oxides of the transition elements such as titaniumoxides, hafnium oxides, iridiumoxides, chromium oxides, and aluminum oxides. Silicon based materials may also be used.

Moreover, the structural elements could be fabricated from the biologically active material or any other biodegradable material such as polyelectrolyte biodegradable shells. The types of biodegradable material that are used can be selected based on the drugs used as well as the desired time of release of the drug. In a specific embodiment, the biodegradable material for the structural element is a quick dissolving polysaccharide (such as sugar crystals). The polysaccharide may be used to release heparin. If a release time is desired, for example, for the release of drugs such as taxol, biodegradable polyesters may be used as a biodegradable material for the structural elements.

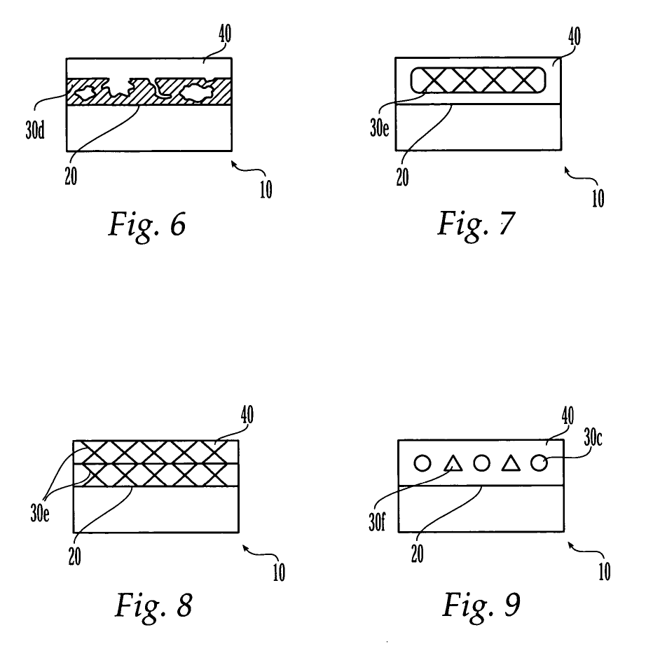

FIG. 6 shows a perspective view of a stent or medical device 10 having a surface 20 wherein the surface 20 is covered with a coating layer 40 wherein porous structural elements 30d are embedded in the coating layer 40. An example of a porous material that could be used as a structural element 30d is a mesoporous or nanoporous ceramic. Biologically active agents not only can be carried by the coating layer 40 but may also be introduced into the pores of structural elements 30d made of porous material. It is also possible to optionally fill the porous structures with a biodegradable substance that would delay the release of the biologically active agent from the pores. Suitable biodegradable substances for this purpose include, but are not limited to, a polysaccharide or a heparin.

In certain embodiments, the structural elements are biodegradable structural elements that have a hardness that is greater than that of the coating layer material. These structural elements comprise biologically active material. Such material is released at least in part by compression of the coating layer, i.e., the release rate of the biologically active material is based at least in part on the amount of compression applied to the coating layer. When the coating layer comprising such structural elements is compressed, the biologically active material will be released. Since the structural elements comprise a biodegradable material, more drugs can be released as compared to structural elements that do not comprise a biodegradable material.

Moreover, if a plurality of structural elements are disposed on the medical device 10 at least two of the structural elements may be interconnected. FIGS. 7 and 8, as alternate embodiments of the present invention, show interconnected structural elements 30e embedded in the coating layer 40 of the medical device 10. FIG. 7 specifically shows one embodiment of the present invention where the interconnected structural elements 30e are surrounded by the coating layer material. FIG. 8 shows another embodiment of the present invention wherein the interconnected structural elements 30e are disposed on the surface 20 of the medical device 10. The interconnected structural elements 30e may form a lattice network of any material, such as a network of stainless steel fibers, bucky paper, or a porous ePTFE sheath.

Structural elements can also be a variety of shapes such as, but not limited to, spheres, shells, discs, rods, struts, rectangles, cubics, oblique spheroids, triangles, pyramidals, tripods, or matrices, or a combination thereof. FIGS. 3 and 4 show an embodiment of spherical structural elements 30c. FIG. 9 depicts a combination of triangular and spherical shaped structural elements 30c, 30f. FIGS. 7 and 8 depict interconnected structural elements 30e in the shape of a matrix.

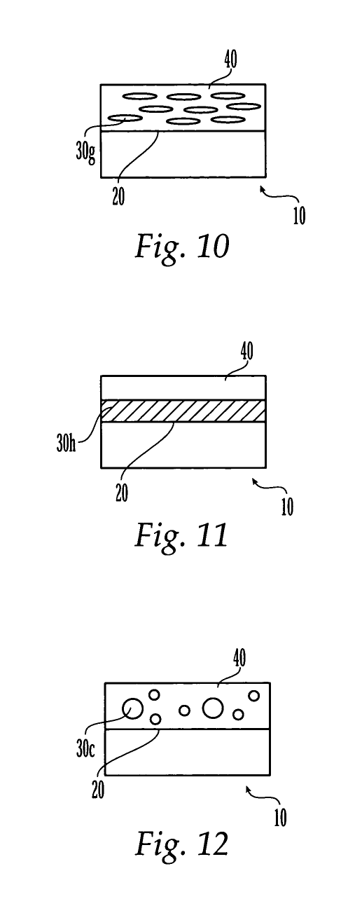

Moreover, the structural elements can be homogeneous i.e., the structural element has the same chemical or physical properties through the entire structural elements. Also, the structural element can be multi-sectioned in which the structural element exists as sections having different chemical or physical properties. For example, a structural element can be made of a ceramic core with an overlaying electrolyte shell. The structural elements can also be multi-layered i.e. have more than one layer. The structural elements may also be disposed evenly or unevenly in the coating layer 40. FIG. 10 depicts a plurality of disc-shaped structural elements 30g disposed evenly or uniformly in a coating layer 40. In certain embodiments, the structural elements are ceramic particles. Optionally, the ceramic particle is covered with an electrolyte shell.

The structural elements suitable for the invention may be any size or height. Preferably, the structural element has a height that is no greater than the thickness of the coating layer 40. For example, the structural elements may only be a percentage of the height of the coating layer 40. In certain embodiments the height or thickness of the structural element is at most 100%, 99%, 95%, 90%, 85%, 80%, 75%, 70%, 65%, 60% 55%, 50%, 45%, 40%, 35%, 30%, 25%, 20%, 15%, 10% or 5% of the thickness of the coating layer containing the structural elements.

FIG. 11 shows the structural element 30h at 50% of the height of the coating layer 40. When the coating layer 40 is compressed, the coating layer 40 may be compressed to 50% of its non-compressed state. The height of the structural elements can be chosen so that the height or thickness of the coating layer 40 when a compression force is applied to the coating layer 40 is a certain percentage of the thickness of the coating layer 40 absent the compression force. By controlling the amount in which the coating layer 40 can be compressed the release rate of a biologically active agent can be controlled. For example, the structural elements may be designed to minimize compression of the coating layer 40 and thereby prevent an initial release of the biologically active material and achieve a sustained release of the biologically active material. The structural elements may also be designed to allow the coating layer 40 to be compressed a certain amount to cause an initial release of the biologically active material followed by a continuous release through diffusion.

Furthermore, the structural elements in the coating layer 40 may vary in size. In one embodiment, represented in FIG. 12, the structural elements 30c are various sized spheres.

The structural elements may be positioned in any desired pattern or distribution on the medical device 10. For example, when the medical device is a stent, the structural elements may be disposed on the outer surface of the stent, the inner surface of the stent, the side surfaces such as between the struts of a stent, or any combination thereof.

The structural elements may be embedded into the coating layer using any suitable method. Preferably, the structural elements are applied to the medical device at the same time that the coating layer is formed, such as by pre-mixing the structural elements with the coating composition and applying the coating composition onto the surface of the medical device to form the coating layer with the structural elements embedded therein.

The structural elements may also be applied after the coating layer has been formed on the surface of the medical device. For instance, the structural elements may also be embedded into the coating layer using electrostatic forces as disclosed in co-pending application Ser. No. 10/335,510 to Weber, filed Dec. 30, 2002. Another method for introducing structural elements into the coating layer is to place the structural elements into the coating layer using nano-robots or other production systems that provide micro- or nano-scale precision which are commercially available. For example, placement systems manufactured by Klocke Nanotechnik of Germany may be used. Still another method for disposing the structural elements into the coating layer is to form cavities in the coating layer of the medical device and then insert the structural elements into the cavities. The cavities may be formed by laser ablation. In this embodiment, the structural elements may comprise a porous material, and the biologically active material may be contained within the pores of the structural element.

The structural elements may be disposed on the surface of the medical device before the coating composition is applied. One method of disposing the structural elements on to the surface of the medical device is to manufacture the medical device using a mold that already includes the structural elements on the surface. Another method is to weld the structural elements on to the surface of the medical device. Still another method is to etch the structural elements out of the surface of the medical device using a laser. A further method includes applying a polymer layer onto the surface of the medical device then using laser ablation to create a pattern in the first polymer. The pattern can function as the structural elements. A second polymer that is softer than the first polymer is applied over or around at least part of the pattern to form a coating layer. Additionally, an inkjet printer may be used to position the hard polymer structures prior to depositing the softer topcoating layer.

In one method of forming the aforementioned coating layers, a coating material composition is applied to the surface. Coating compositions can be applied by any method to a surface of a medical device to form a coating layer. Examples of suitable methods include, but are not limited to, spraying such as by conventional nozzle or ultrasonic nozzle, dipping, rolling, electrostatic deposition, and a batch process such as air suspension, pancoating or ultrasonic mist spraying. Also, more than one coating method can be used to make a medical device. Coating compositions suitable for applying a coating to the devices of the present invention can include a polymeric material dispersed or dissolved in a solvent suitable for the medical device, wherein upon applying the coating composition to the medical device, the solvent is removed. Such systems are commonly known to the skilled artisan.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material are styrene-isobutylene-styrene copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

Preferably, for medical devices which undergo mechanical challenges, e.g., expansion and contraction, polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

Solvents used to prepare coating compositions include ones which can dissolve or suspend the polymeric material in solution. Examples of suitable solvents include, but are not limited to, tetrahydrofuran, methylethylketone, chloroform, toluene, acetone, isooctane, 1,1,1,-trichloroethane, dichloromethane, isopropanol, IPA, and mixture thereof.

The medical device coating layer may also contain one or more biological active materials. A biologically active material can also be included in the structural element. The term “biologically active material” encompasses therapeutic agents, such as biologically active agents, and also genetic materials and biological materials. The genetic materials mean DNA or RNA, including, without limitation, of DNA/RNA encoding a useful protein stated below, intended to be inserted into a human body including viral vectors and non-viral vectors as well as anti-sense nucleic acid molecules such as DNA, RNA and RNAi. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor and platelet derived endothelial growth factor, platelet derived growth factor, tumor necrosis factor, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as:

anti-thrombogenic agents such as heparin, heparin derivatives, urokinase, and PPack (dextrophenylalanine proline arginine chloromethylketone);

anti-proliferative agents such as enoxaprin, angiopeptin, geldanamycin, or monoclonal antibodies capable of blocking smooth muscle cell proliferation, hirudin, acetylsalicylic acid, tanolimus, everolimus, amlodipine and doxazosin;

anti-inflammatory agents such as glucocorticoids, betamethasone, dexamethasone, prednisolone, corticosterone, budesonide, estrogen, sulfasalazine, rosiglitazone, mycophenolic acid, and mesalamine;

antineoplastic/antiproliferative/anti-miotic agents such as paclitaxel, 5-fluorouracil, cisplatin, vinblastine, vincristine, epothilones, epithilone D, methotrexate, azathioprine, adriamycin and mutamycin; endostatin, angiostatin and thymidine kinase inhibitors, cladribine, taxol and its analogs or derivatives;

anesthetic agents such as lidocaine, bupivacaine, and ropivacaine;

anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, antiplatelet agents such as trapidil or liprostin, platelet inhibitors and tick antiplatelet peptides;

vascular cell growth promotors such as growth factors, Vascular Endothelial Growth Factors (FEGF, all types including VEGF-2), growth factor receptors, transcriptional activators, and translational promotors;

DNA demethylating drug such as 5-azacytidine, which is also categorized as a RNA or DNA metabolite that inhibit cell growth and induce apoptosis in certain cancer cells;

vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;

cholesterol-lowering agents; vasodilating agents; and agents which interfere with endogenous vasoactive mechanisms;

anti-oxidants, such as probucol;

antibiotic agents, such as penicillin, cefoxitin, oxacillin, tobranycin, rapamycin (sirolimus);

antagonist for collagen synthesis, such as halofuginone;

angiogenic substances, such as acidic and basic fibrobrast growth factors, estrogen including estradiol (E2), estriol (E3) and 17-Beta Estradiol;

anti-platelet aggregation substance, phosphodiesterase inhibitors, such as cilostazole;

smooth muscle cell proliferation inhibitors, such as rapamycin; and

drugs for heart failure, such as digoxin, beta-blockers, angiotensin-converting enzyme (ACE) inhibitors including captopril and enalopril, statins and related compounds.

Preferred biologically active materials include anti-proliferative drugs such as steroids, vitamins, and restenosis-inhibiting agents. Preferred restenosis-inhibiting agents include microtubule stabilizing agents such as paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred biologically active materials include nitroglycerin, nitrous oxides, nitric oxides, antibiotics, aspirins, digitalis, estrogen derivatives such as estradiol and glycosides.

The biologically active material may also be applied with a coating composition. Coating compositions suitable for applying biologically active materials to the devices of the present invention preferably include a polymeric material and a biologically active material dispersed or dissolved in a solvent which does not alter or adversely impact the therapeutic properties of the biologically active material employed. Suitable polymers and solvents include, but are not limited to, those listed above.

In another embodiment, the coating composition comprises a non-polymeric material. In another embodiment, the coating composition comprises entirely of biologically active material. In a specific embodiment, embedded in the biologically active material coating are a plurality of structural elements.

Coating compositions may be used to apply one type of biologically active material or a combination of biologically active materials. In general, the coating layer may be applied as one homogeneous layer, however, as in FIG. 3, the coating layer 60 may be composed of a plurality of layers 42, 44, 46, 48 comprised of different materials. If the coating layer is composed of a plurality of layers, each layer may contain a single biologically active material or a combination of biologically active materials.

A biologically active material may be delivered to a body lumen using the medical device described above. The stent, or other medical device, is inserted into body of the patient by a method known to artisan. For example, when the stent of the present invention is a self-expandable stent, then the stent is collapsed to a small diameter by placing it in a sheath, introduced into a lumen of a patient's body using a catheter, and is allowed to expand in the target area by removing it from the sheath. When the stent of the present invention is a balloon expandable stent, the stent is collapsed to a small diameter, placed over an angioplasty balloon catheter, and moved into the area to be placed. When the balloon is inflated, the stent expands.

The method of the present invention has many advantages including providing an efficient, cost-effective, and relatively safe manufacturing process for applying a biologically active material to a medical device. The present method provides a medical device having a coating layer that is reasonably durable and resistant to the compressive forces applied to the coating layer during delivery and implantation of the medical device, and offers some control over the release rate of a biologically active material from the coating layer.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein by reference, in their entirety, for all purposes related to this disclosure.

What is claimed is:

1. A coated medical device for delivering a biologically active material to body tissue comprising:
a medical device having a surface; and
a coating layer disposed on at least a portion of the surface, wherein the coating layer comprises a biocompatible polymer and at least one structural element embedded in the coating layer, wherein the structural element reduces the compressibility of the coating layer.

2. The medical device of claim 1, wherein the polymer has a first hardness and the structural element has a second hardness that is greater than the first hardness.

3. The medical device of claim 1, wherein the structural element comprises a metal, a ceramic, a polymer, or a biologically active material.

4. The medical device of claim 1, wherein the structural element comprises a porous material.

5. The medical device of claim 1, wherein the structural element comprises more than one layer.

6. The medical device of claim 1, wherein the structural element comprises a biodegradable material.

7. The medical device of claim 6, wherein the structural element is a polyelectrolyte biodegradable shell.

8. The medical device of claim 1, wherein a plurality of structural elements are embedded in the coating layer and at least two of the structural elements are interconnected.

9. The medical device of claim 1, wherein the structural element is configured in a shape comprising a sphere, a shell, a disc, a rod, a strut, a rectangle, an oblique spheroid, a cube, a triangle, a pyramid, a tetrahedron, a matrix, a wire network or a crosslinked microvolume.

10. The medical device of claim 1, wherein the medical device is a balloon catheter.

11. The medical device of claim 1, wherein the medical device is a stent.

12. The medical device of claim 1, wherein the coating layer comprises at least one biologically active material.

13. The medical device of claim 12, wherein the biologically active material comprises an anti-thrombogenic agent, an anti-angiogenesis agent, an anti-proliferative agent, a growth factor, a radioactive chemical or combinations thereof.

14. The medical device of claim 13, wherein the anti-proliferative agent comprises paclitaxel, a paclitaxel analogue, a paclitaxel derivative, or combinations thereof.

15. The medical device of claim 1, wherein the structural element is in contact with the device surface.

16. A coated stent for delivering a biologically active material to body tissue comprising:
a stent having a surface; and
a coating layer disposed on at least a portion of the surface, wherein the coating layer comprises a biocompatible polymer, a biologically active material and a plurality of structural element embedded in the coating layer; wherein the structural elements reduce the compressibility of the coating layer.

17. A coated stent for delivering a biologically active material to body tissue comprising:
a stent having a surface; and
a coating layer disposed on at least a portion of the surface, wherein the coating layer comprises a biocompatible polymer having a first hardness, a first biologically active material and a plurality of structural elements embedded in the coating layer; wherein the structural elements reduce the compressibility of the coating layer and wherein the structural elements comprise a porous material having a second hardness, and whereby the first hardness is less than the second hardness.

18. The coated stent of claim 17, wherein the structural elements comprise a second biologically active material.

19. The coated stent of claim 17, wherein the structural elements comprise a ceramic material ceramic.

20. A coated medical device comprising:
a medical device having a surface; and
a coating layer disposed on at least a portion of the surface, wherein the coating layer comprises a biocompatible non-polymeric material and at least one structural element embedded in the coating layer, wherein the structural element reduces the compressibility of the coating layer.

21. The medical device of claim 20, wherein the structural element comprises a metal, a ceramic, a polymer, or a biologically active material.

22. The medical device of claim 20, wherein the structural element comprises a biodegradable material.

23. The medical device of claim 20, wherein a plurality of structural elements are embedded in the coating layer and at least two of the structural elements are interconnected.

24. The medical device of claim 20, wherein the structural element is configured in a shape comprising a sphere, a shell, a disc, a rod, a strut, a rectangle, an oblique spheroid, a cube, a triangle, a pyramid, a tetrahedron, a matrix, a wire network or a crosslinked microvolume.

25. The medical device of claim 20, wherein the medical device is a balloon catheter.

26. The medical device of claim 20, wherein the medical device is a stent.

27. The medical device of claim 20, wherein the coating layer comprises at least one biologically active material.

28. A coated medical device comprising:
a medical device having a surface; and
a coating layer disposed on at least a portion of the surface, wherein the coating layer comprises a biologically active material and at least one structural element embedded in the coating layer, wherein the structural element reduces the compressibility of the coating layer.

29. The medical device of claim 28, wherein the structural element comprises a metal, a ceramic, a polymer, or a biologically active material.

30. The medical device of claim 28, wherein the structural element comprises a biodegradable material.

31. The medical device of claim 28, wherein a plurality of structural elements are embedded in the coating layer and at least two of the structural elements are interconnected.

32. The medical device of claim 28, wherein the structural element is configured in a shape comprising a sphere, a shell, a disc, a rod, a strut, a rectangle, an oblique spheroid, a cube, a triangle, a pyramid, a tetrahedron, a matrix, a wire network or a crosslinked microvolume.

33. The medical device of claim 28, wherein the medical device is a balloon catheter.

34. The medical device of claim 28, wherein the medical device is a stent.

35. A method of using the medical device of claim 1 comprising applying a predefined amount of a compressive force to the coating layer.

36. The method of claim 35 wherein application of the predefined amount of the compressive force affects the release rate of a biologically active material included in the coating layer.
